(19) United States
(12) Patent Application Publication
(10) Pub. No.: US 2017/0224615 A1
(43) Pub. Date: Aug. 10, 2017

(54) BONE AND LIGAMENT STIMULATING NON-STICK GUM

(71) Applicant: Ortho Gum, Inc., Denver, CO (US)

(72) Inventors: Amy Carpenter, Denver, CO (US); William P. Paini, Denver, CO (US)

(73) Assignee: Ortho Gum, Inc., Denver, CO (US)

(21) Appl. No.: 15/421,857

(22) Filed: Feb. 1, 2017

Related U.S. Application Data
(60) Provisional application No. 62/291,051, filed on Feb. 4, 2016.

(51) Int. Cl. A61K 9/68; A23G 4/06; A61C 7/00; A61K 8/02; A61Q 11/00; A23G 4/08; A61K 31/047

(52) U.S. Cl. CPC A61K 9/0058; A23G 4/08; A23G 4/06; A61K 31/047; A61K 8/0216; A61Q 11/00; A61C 7/00; A23V 2002/00; A61K 2800/92

(57) ABSTRACT

A chewing gum composition that can be used by patients using removable orthodontic appliances is provided. The chewing action provided by the gum composition stimulates bones and ligaments which helps the removable orthodontic appliance move the patient's teeth in a predetermined fashion.

This application claims the benefit of U.S. Provisional Application No. 62/291,051, filed Feb. 4, 2016, the entirety of which is incorporated by reference herein.

### FIELD OF THE INVENTION

Embodiments of the present invention generally relate to a chewing gum compound that stimulates a patient's bones and/or ligaments to accelerate orthodontic movements. The chewing gum compound of one embodiment will not stick to dental restorations, orthodontic appliances, or dental appliances.

### BACKGROUND OF THE INVENTION

Correcting misaligned teeth is usually accomplished by applying braces comprised of metal brackets interconnected to patient's upper or lower teeth. The plurality of metal brackets are interconnected to each other by way of a wire that is periodically adjusted to slowly force the teeth into a desired alignment. Traditional braces are often uncomfortable as the metal brackets and wire impinge against the inner surface of the patient's mouth. Traditional braces also trap food particles, which may cause tooth decay or bad breath. For some patients, the most important drawback is that traditional braces are unsightly, so many patients avoid getting braces which exasperates tooth misalignment.

Because of these and other drawbacks associated with traditional braces, clear, removable dental appliances have become a very popular alternative to treating tooth misalignment. Removable dental appliances fit snugly onto a patient's teeth and precisely move the teeth to a desired position as appliances are periodically modified and replaced.

Although more appealing because they are normally clear, removable orthodontic appliances also have drawbacks. For example, removable orthodontic appliances often cause pain as the teeth are moved to the correct positions. Although fixed orthodontic appliances also have this drawback, a patient cannot easily remove their braces to find relief. Conversely, a patient can remove an orthodontic appliance to achieve instant satisfaction, which can extend tooth adjustment time as it is preferred that the removable orthodontic appliance be positioned within the mouth continuously.

Another drawback of removable dental appliances is they must be tightly seated on the patient's teeth. That is, the tolerance between the inner profile of the dental appliance and the outer surface of the patient's teeth is designed to be very tight to facilitate tooth adjustment. Some patients require seating devices to firmly attach the removable orthodontic appliance to their teeth. Unfortunately, such devices are sometimes unhygienic.

Chewing activities facilitate tooth movement by stimulating the bones and ligaments associated with a patient's teeth. Research shows that chewing may stimulate tooth movement, as jaw hypofunction leads to lower mineral apposition and bone function; and narrow periodontal ligament (PDL). These conditions may lead to impaired remodeling process, and increases the susceptibility of root resorption during orthodontic tooth movement. Moreover, as stimulation of the PDL could be mechanoreceptive (i.e. chewing action) or nociceptive (i.e. painful stimulation), periodontal nerve fibers are supposed to play an important role in bone remodeling (see: “A new concept in orthodontics: faster and healthier tooth movement by regularly consuming Xylitol chewing gum by Haryono Utomo. 12/2007.)

Again, decreasing the time a patient must use any orthodontia is desired. Regrettably, chewing some foodstuffs, e.g., chewing gum, has been impossible as such compounds are often sticky and adhere to removable orthodontic appliances, which can cause them to separate from the patient's teeth. Tacky substances adhering to removable orthodontic appliances will also attract food, which could cause bad breath.

Conventional chewing gum compounds often comprise elastomer solvents and other sticky ingredients. Elastomer solvents allow the chewing gum compound to exhibit a continuous or substantially continuous phase when chewed. See, for example, U.S. Pat. No. 4,518,615 to Cherukuri and U.S. Patent Application Publication No. 2012/0045494 to Harris et al. for a description of traditional chewing gum compounds. As articulated in Harris, many elastomers used in chewing gum compositions are solid at room temperature and require solvents to make them soft enough to be useful in chewing gum compositions. Traditional solvents have the drawback of making the gum composition tacky.

It is a long-felt need in dentistry to provide a chewing gum compound that can be used with removable orthodontic appliances. Embodiments of the present invention are directed to chewing gum base compositions that do not here to dentures, removable and fixed oral orthodontic devices, fillings, or natural teeth.

### SUMMARY OF THE INVENTION

It is another aspect of embodiments of the present invention to provide a chewing gum compound that does not stick to orthodontic appliances, such as braces (traditional brackets and bands), clear orthodontic aligners, orthodontic appliances, and orthodontic retainers. The contemplated gum will not stick to dental restorations, such as crowns, fillings, prosthetic retainers, or dentures. That is, the gum of one embodiment possesses a decreased adhesive index, which results in a composition that does not stick to removable orthodontic appliances. The composition of one embodiment has an adhesive index less than that of chewing gum, which allows use of the compound while the orthodontic appliances are positioned in a patient's mouth. The chewing action stimulates surrounding bones and ligaments to accelerate tooth movement by a removable orthodontic appliance.

It is another aspect of embodiments the present invention to provide a gum compound of a precise hardness. More specifically, as described above, chewing action helps stimulate bone and ligaments of the mouth which facilitates tooth movement. If a gum compound is too soft, it will not achieve the desired stimulation. And, if the gum compound is too hard, it will be uncomfortable to chew. Accordingly, one embodiment present invention has a hardness of about 50 to about 80 Shore OO. Another embodiment has a hardness of about 10 to about 50 Shore A. Yet another embodiment of the present invention has a hardness corresponding to that of a wax cube as articulated in “Objective and Subjective Hardness of a Test Item used for Evaluate for Mixing Ability,” Salleh, et al., Journal of Oral Rehabilitation, Vol. 34, pages 174-183 (2007).

It is another aspect of the present invention to provide a gum that reduces bacteria that can grow inside the mouth or on the removable orthodontic appliance. More specifically, the gum composition of some embodiments of the present invention includes xylitol, a known bacteria fighter. Xylitol is also known to help balance mouth pH, and it is sugar-free and anti-cariogenic. These features of xylitol also help to clean the patient's mouth and the removable orthodontic appliance. Plaque buildup is also decreased when Xylitol is used, which creates a healthier oral environment.

It is still yet another aspect of some embodiments of the present invention to provide a gum that helps seat orthodontic aligners. As mentioned above, and the hardness of the gum makes it ideal for chewing, and it's stick-free nature makes it ideal to be used with removable orthodontic appliances. And associated benefit is that the chewing action helps seat the removal orthodontic appliances and maintains the position in the patient's mouth. Promoting chewing action is also known to decrease pain when transitioning from a removable orthodontic appliance to the next.

The Summary of the Invention is neither intended nor should it be construed as being representative of the full extent and scope of the present invention. That is, these and other aspects and advantages will be apparent from the disclosure of the invention(s) described herein. Further, the above-described embodiments, aspects, objectives, and configurations are neither complete nor exhaustive. As will be appreciated, other embodiments of the invention are possible using, alone or in combination, one or more of the features set forth above or described below. Moreover, references made herein to “the present invention” or aspects thereof should be understood to mean certain embodiments of the present invention and should not necessarily be construed as limiting all embodiments to a particular description. The present invention is set forth in various levels of detail in the Summary of the Invention as well as in the attached drawings and the Detailed Description of the Invention and no limitation as to the scope of the present invention is intended by either the inclusion or non-inclusion of elements, components, etc. in this Summary of the Invention. Additional aspects of the present invention will become more readily apparent from the Detail Description, particularly when taken together with the drawings.

### DETAILED DESCRIPTION

Embodiments of the present invention is comprised of at least the following ingredients:

| | |
|---|---|
| Dental non-stick base | X % |
| Xylitol | X % |
| Citric Acid | X % |
| Titanium dioxide | X % |
| Mint flavoring | X % |
| Peppermint Aroma Oil | X % |
| Peppermint powder | X % |
| Water | X % |
| Calcium Stearate | X % |

Strawberry, watermelon, peppermint, winter mint, spearmint, or any other flavors deemed may be added to the base gum compound to satisfy market needs.

In operation, the patient would chew one or two pieces of gum for about 10 minutes to help to simulate the bones and ligaments associated with the teeth being repositioned. Patients should not exceed 10 minutes of use is doing so may cause undue stress in the temporomandibular joint.

While various embodiments of the present invention have been described in detail, it is apparent that modifications and alterations of those embodiments will occur to those skilled in the art. It is to be expressly understood that such modifications and alterations are within the scope and spirit of the present invention, as set forth in the following claims. Further, it is to be understood that the invention(s) described herein is not limited in its application to the details of construction and the arrangement of components set forth in the preceding description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

What is claimed is:

1. A chewing gum composition comprising, based on the total weight of the composition:
(a) 0.01 to 1% by weight of calcium stearate;
(b) 25 to 85% by weight of a gum base comprising a buffering agent selected from the group consisting of acetates, glycinates, phosphates, carbonates, glycerophosphates, citrates, borates, and mixtures thereof;
(c) 10 to 35% by weight of at least one sweetening agent selected from the group consisting of xylitol, sorbitol, isomalt, aspartame, sucralose, acesulfame potassium, and saccharin;
(d) 1 to 10% by weight of flavoring agents selected from the group consisting of menthol flavor, eucalyptus, mint flavor and/or L-menthol.

2. The chewing gum composition according to claim 1, wherein the chewing gum composition comprises a pharmaceutically acceptable excipient selected from the group consisting of fillers, disintegrants, binders, lubricants, and antioxidants.

3. The chewing gum composition according to claim 1, wherein the chewing gum composition is non-disintegrating.
